(19) United States
(12) Patent Application Publication
(10) Pub. No.: US 2026/0273186 A1
(43) Pub. Date: Sep. 17, 2026

(54) Medicament delivery device

(71) Applicant: SHL Medical AG, Zug (CH)

(72) Inventors: Ming-Ting Yin, Taoyuan (TW); Hamza Azam, Taoyuan City (TW); Lucas Carpenter, Taoyuan (TW); Yi-Wen Lo, Taoyuan City (TW); Shang Yi Yi, Taoyuan City (TW)

(21) Appl. No.: 19/166,107

(22) PCT Filed: Mar. 7, 2024
(86) PCT No.: PCT/EP2024/056005

§ 371 Date: Sep. 17, 2025

(30) Foreign Application Priority Data
Mar. 21, 2023 (EP) 23163002.1

(51) Int. Cl. A61M 5/178; A61M 5/31

(52) U.S. Cl. CPC A61M 5/178; A61M 2005/312

(57) ABSTRACT

The present disclosure provides a medicament delivery device including a housing extending along a longitudinal axis. The medicament delivery device further includes a rear cap closing a rear opening at the distal end of the housing, the rear cap including a through slot extending orthogonal to the longitudinal axis, the slot comprising diametrically opposite first and second access holes. The medicament delivery device further includes a tray comprising a set of electronic components, arranged in the slot of the rear cap. A distal portion of the housing comprises a lateral opening facing the first access hole of the slot of the rear cap, through which the tray can be slid out of the slot.

### TECHNICAL FIELD

The present disclosure relates to a medicament delivery device.

### BACKGROUND

Medicament delivery devices such as pen-type manual injectors or auto-injectors are generally known for the self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users have no professional training or are in an emergency situation, the users can easily and properly use the medicament delivery devices. Those users who have no professional training might need more instructions/indications to understand how to use the medicament delivery devices properly. Therefore, it may be useful to incorporate a module including a set of electronic components, for instance a battery, sensors, indicators and/or data receiving/transmitting means to the medicament delivery devices to provide instructions/indications to those users.

However, in the current medicament delivery devices having a module including a set of electronic components, such a module is difficult to extract for recycling and/or refurbishment reasons.

### SUMMARY

This need is met by a medicament delivery device according to the present disclosure.

The invention is specified by the independent claims. Preferred embodiments are defined in the dependent claims. In the following description, although numerous features may be designated as optional, it is nevertheless acknowledged that all features comprised in the independent claims are not to be read as optional.

According to a first embodiment, the invention relates to a medicament delivery device, comprising:
a housing extending along a longitudinal axis,
a rear cap closing a rear opening at the distal end of the housing, the rear cap comprising a through slot extending orthogonal to the longitudinal axis, the slot comprising diametrically opposite first and second access holes,
a tray comprising a set of electronic components, arranged in the slot of the rear cap,
   wherein
a distal portion of the housing comprises a lateral opening facing the first access hole of the slot of the rear cap, through which the tray can be slid out of the slot.

Optionally, the tray comprises a wall closing the lateral opening of the housing.

Optionally, the housing comprises at least one cut-out connected to the lateral opening of the housing, said cut-out forming an extraction hole through which an extraction tool can be inserted to slide the tray out of the slot.

Optionally, the distal portion of the housing comprises an extraction hole diametrically opposite the lateral opening of the housing and facing the second access hole of the slot of the rear cap, through which an extraction tool can be inserted to push the tray out of the slot.

Optionally, the lateral opening and the rear opening are connected to each other at the distal end of the housing.

Optionally, the slot is distally delimited by a distal surface of the rear cap that closes the rear opening of the housing.

Optionally, the slot is proximally delimited by a support plate at a distal extremity of a tubular body of the rear cap.

Optionally, the rear cap is fixedly attached to the distal portion of the housing.

Optionally, the rear cap is fixedly attached to the distal portion of the housing by an engagement of a pair of flexible tongues of the housing with a pair of corresponding ledges of the rear cap.

According to a second embodiment, the invention relates to a medicament delivery device, comprising:
a housing extending along a longitudinal axis,
a rear cap closing a rear opening at the distal end of the housing, the rear cap comprising a through slot extending orthogonal to the longitudinal axis, the slot comprising diametrically opposite first and second access holes,
a tray comprising a set of electronic components, arranged in the slot of the rear cap,
   wherein
the rear cap comprises a distal part and a proximal part removably attached to each other, the distal part having a distal surface closing the rear opening of the housing,
the slot is arranged in the distal part of the rear cap.

Optionally, the distal surface has at least an extraction hole in which an extraction tool can be inserted to detach the distal part from the proximal part in axial direction.

Optionally, the distal part and the proximal part of the reap cap are screwed to each other, and the distal surface of the distal part has two extraction holes in which an extraction tool can be inserted to unscrew the distal part from the proximal part in axial direction.

Optionally, the two extraction holes are diametrically opposite relative to the longitudinal axis.

Optionally, the proximal part of the rear cap is fixedly attached to a distal portion of the housing.

Optionally, the proximal part of the rear cap is fixedly attached to the distal portion of the housing by an engagement of a pair of flexible tongues of the housing with a pair of corresponding ledges of the proximal part of the rear cap.

Optionally, the slot is distally delimited by the distal surface of the distal part of the rear cap.

Optionally, the slot is proximally delimited by a support plate at a proximal extremity of the distal part of the rear cap.

According to a third embodiment, the invention relates to a medicament delivery device, comprising:
a housing extending along a longitudinal axis,
a rear cap closing a rear opening at the distal end of the housing, the rear cap comprising a through slot extending orthogonal to the longitudinal axis, the slot comprising diametrically opposite first and second access holes,
a tray comprising a set of electronic components, arranged in the slot of the rear cap,
   wherein
the rear cap comprises a main body and a lid removably attached to each other, the lid having a top surface closing at least partially the rear opening of the housing,
the slot is distally delimited by the top surface of lid.

Optionally, a distal portion of the housing comprises at least an extraction hole through which an extraction tool can be inserted to interact with the lid and detach the lid from the main body.

Optionally, the lid comprises two hooks, each hook being engaged in a recess of the main body, and the housing comprises two extraction holes through which an extraction tool can be inserted to disengage the hooks from the recesses.

Optionally, the lid comprises two flexible arms, each ending with a hook, the arms extending parallel to the longitudinal axis, the slot being laterally delimited by the arms.

Optionally, the slot is proximally delimited by a support plate at a distal extremity of a tubular body of the main body of the rear cap.

Optionally, the main body of the rear cap is fixedly attached to a distal portion of the housing.

Optionally, the main body of the rear cap is fixedly attached to the distal portion of the housing by an engagement of a pair of flexible tongues of the housing with a pair of corresponding ledges of the main body of the rear cap. In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a perspective view of a medicament delivery device according to a first embodiment of the invention.

FIGS. 2 and 3 show perspective views of a rear cap according to the first embodiment.

FIG. 4 shows a perspective view of a tray according to the first embodiment.

FIG. 5 shows a perspective view of the rear cap of FIGS. 2 and 3, hosting the tray of FIG. 4.

FIG. 6 shows a distal portion of a housing according to the first embodiment.

FIG. 7 shows a perspective view of a medicament delivery device according to a second embodiment of the invention.

FIG. 8 shows a perspective view of a rear cap according to the second embodiment.

FIG. 9 shows a perspective view of a tray according to the second embodiment.

FIG. 10 shows a perspective view of the rear cap of FIG. 8, hosting the tray of FIG. 9.

FIG. 11 shows a perspective view of a medicament delivery device according to a third embodiment of the invention.

FIG. 12 shows a perspective view of a rear cap according to the third embodiment.

FIG. 13 shows a perspective view of a tray according to the third embodiment.

FIG. 14 shows a cross section of a part of the rear cap of FIG. 12, hosting the tray of FIG. 13.

### DETAILED DESCRIPTION

In the figures, elements not essential for carrying out the invention have been omitted, in order not to render the depictions overly complicated and not to blur the scope of the disclosure.

In the following, the term engagement encompasses any kind of interaction between the named features such as abutment, clamping, locking etc.

Three embodiments of a medicament delivery device according to the invention are going be detailed thereafter.

### First Embodiment

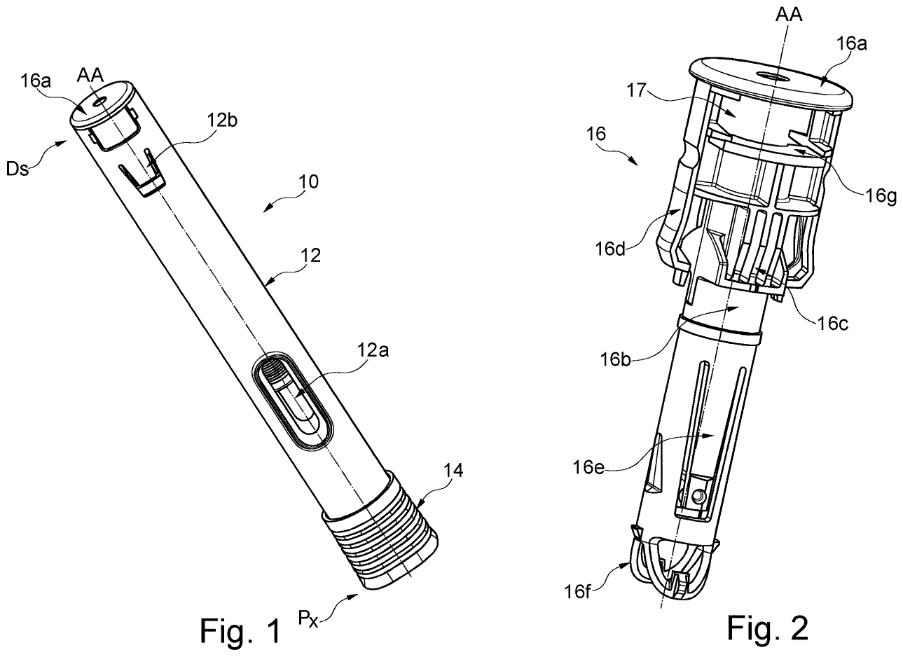

A First Embodiment Is Depicted in FIGS. 1 to 6.

FIG. 1 shows a perspective view of a medicament delivery device 10. The particular medicament delivery device 10 illustrated in FIG. 1 is an injector configured to inject a dose of medicament into the body of a user via a syringe arranged in a housing 12 of the medicament delivery device 10 after a protective front cap 14 has been removed. The medicament delivery device extends along a longitudinal axis AA between a proximal end Px and a distal end Ds, wherein the distal end Ds is pointing away from the user's body during use of the medicament delivery device 10. Concerning the terms “distal” or “rear”, and “proximal” of “front”, they refer to points which are further away and closer to the injection site respectively.

The housing 12 is hollow and may be generally tubular, although other shapes are possible. The housing 12 extends along the longitudinal axis AA between the proximal end Px and the distal end Ds. The housing 12 comprises a circular (if the housing is tubular) front opening (not visible) closed by the front cap 14, and a circular (if the housing is tubular) rear opening 12d (see FIG. 6) closed by a distal surface 16a of a rear cap 16 (the rear cap 16 is shown in FIG. 2). The rear cap 16 is arranged coaxially with the housing 12, and the distal surface 16a of the rear cap 16 extend orthogonally to the longitudinal axis AA.

The housing 12 further comprises an optional window 12a through which the user can see the drug to be injected

The medicament delivery device 10 comprises the rear cap 16, depicted in FIG. 2. The rear cap 16 comprises a tubular body 16b extending along the longitudinal axis AA, the tubular body 16b being arranged coaxially with the housing 12 and arranged radially inside a distal portion of the housing 12.

The rear cap 16 is fixedly attached to the distal portion of the housing 12, preferably by a pair of flexible tongues 12b of the housing 12 engaging with a pair of corresponding ledges 16c on the tubular body 16b.

The rear cap 16 further comprises a pair of flexible arms 16d extending from the distal surface 16a in the proximal direction along the longitudinal axis AA, able to flex from and to the longitudinal axis AA and situated one opposite the other diametrically. The tubular body 16b is equipped with a pair of flexible tongues 16e extending in a proximal direction along the axis AA, able to flex from and to the longitudinal axis AA and situated one opposite the other diametrically. The flexible tongues 16e of the tubular body 16b are displaced on 90° relative to the flexible arms 16 d. Finally, the tubular body 16b is equipped, at its proximal end, with a pair of flexible wings 16f, able to flex from and to the longitudinal axis AA and situated one opposite the other diametrically. The flexible wings 16f of the tubular body 16b are displaced on 90° relative to the flexible arms 16 d.

The function of the flexible arms 16d, the flexible tongues 16e and the flexible 16f are not part of the invention, consequently they will not be described here.

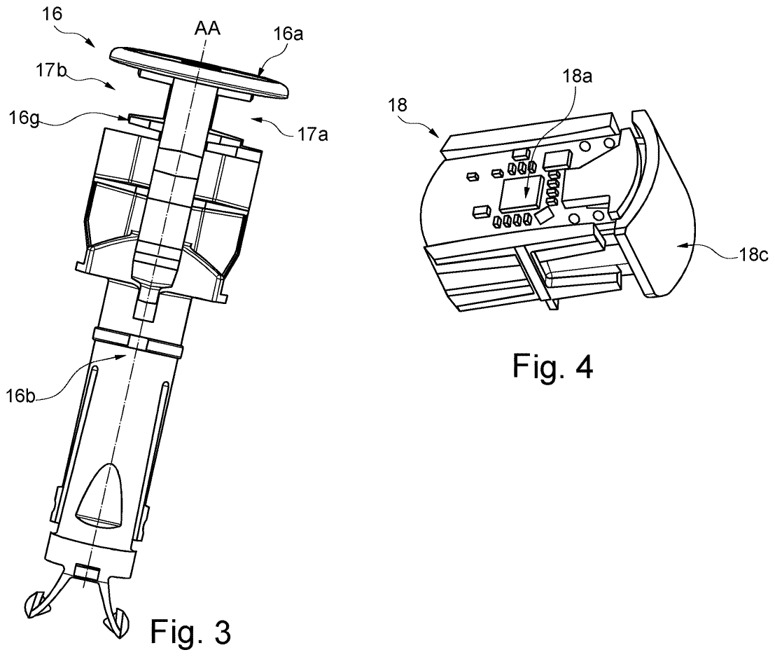

The rear cap 16 further comprises a through slot 17 extending orthogonal to the longitudinal axis AA, the slot 17 comprising two diametrically opposite access holes 17a, 17b (see FIG. 3). A tray 18 (see FIG. 4) can be inserted in the slot 17 and removed from the slot 17 through the access holes 17a, 17b, orthogonally to the longitudinal axis AA.

The slot 17 is axially located between the distal surface 16a and a support plate 16g at a distal extremity of the tubular body 16b. The distal surface 16a and the support plate 16g are supporting elements for the tray 18.

FIG. 4 shows an exemplary tray 18 that can be inserted in the slot 17 of the rear cap 16. The tray 18 comprises a set of electronic components 18a, for instance a battery, sensors, indicators and/or data receiving/transmitting means to the medicament delivery devices to provide instructions/indications to the user.

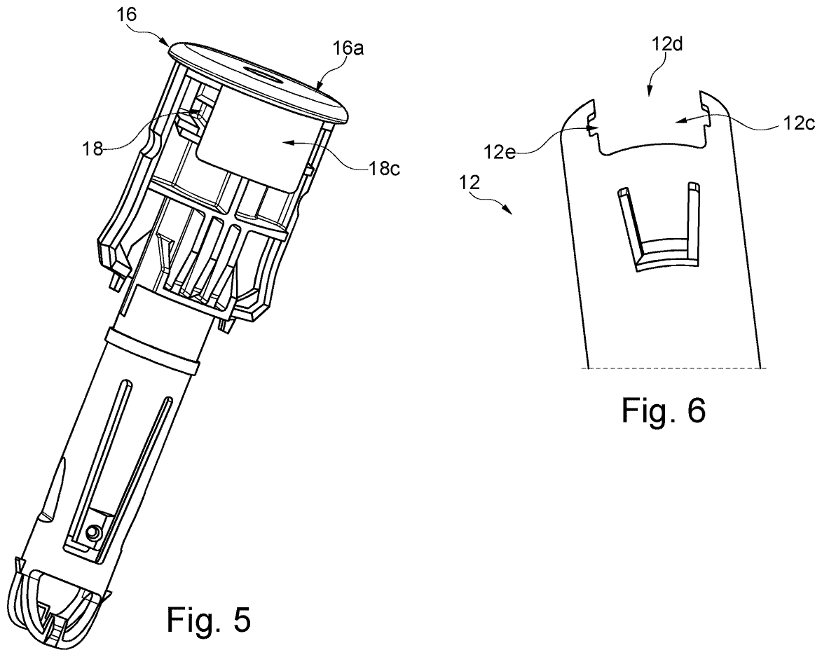

The tray 18 further comprises a wall 18c whose function is to close a first access hole 17a of the slot 17 when the tray 18 is positioned in the slot 17 (see FIG. 5 showing that configuration).

FIG. 6 shows a distal portion of the housing 12. The distal portion of the housing 12 comprises a lateral opening 12c aligned with the first access hole 17a of the slot 17 of the rear cap 16 when the rear cap 16 is in position in the housing 12. It should be noted that the lateral opening 12c of the housing 12 is connected to the rear opening 12d of the housing 12. In another embodiment though, the lateral opening 12c of the housing 12 is disconnected from the rear opening 12d of the housing 12.

The shape and dimensions of the lateral opening 12c of the housing 12 are generally similar to the shape and dimensions of the wall 18c of the tray 18.

Hence, through the lateral opening 12c, the tray 17 can be inserted in and removed from the slot 17 of the rear cap 16 (see FIG. 1 showing a configuration in which the tray is positioned in the slot 17 and the tubular body 16b of the rear cap 16 is positioned in the housing 12, the distal surface 16a of the reap cap 16 closing the rear opening 12c of the housing 12).

In a first option illustrated in FIG. 6, the distal portion of the housing 12 comprises at least one cut-out 12e (two are represented in FIG. 6), the cut-out 12e connected to the lateral opening 12c. Through the cut-out 12e, an extraction tool can be inserted to contact an inner surface of the wall 18 and pull the tray 18 out of the slot 17 of the rear cap 16.

In a second non-illustrated option, the distal portion of the housing 12 comprises an extraction hole diametrically opposite the lateral opening 12c of the housing 12. The extraction hole faces and communicates with the second access hole 17b of the slot 17 of the rear cap 16. Consequently, an extraction tool can be inserted in the extraction hole to push the tray 18 out of the slot 17 of the rear cap 16.

### Second Embodiment

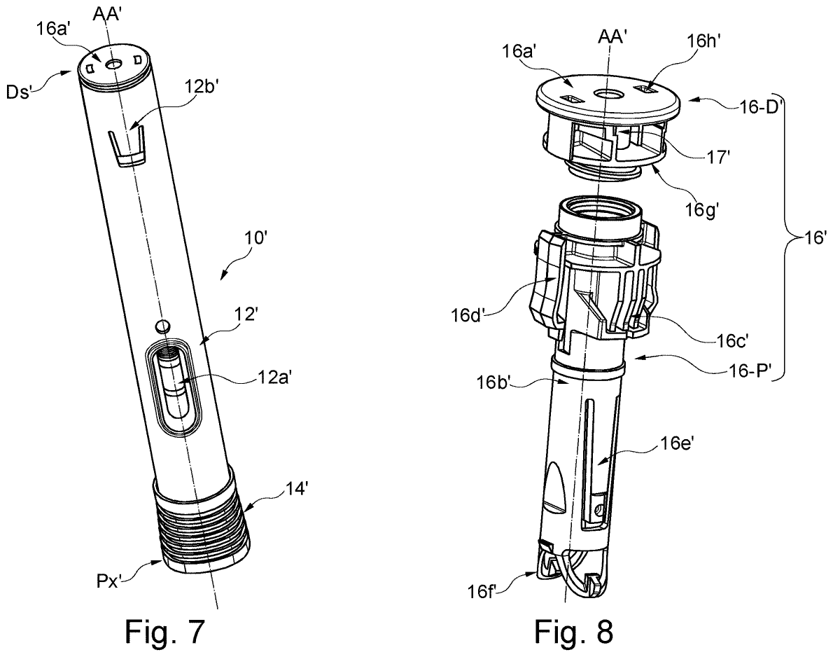

A second embodiment is depicted in FIGS. 7 to 10.

FIG. 7 shows a perspective view of a medicament delivery device 10′. The particular medicament delivery device 10′ illustrated in FIG. 7 is an injector configured to inject a dose of medicament into the body of a user via a syringe arranged in a housing 12′ of the medicament delivery device 10′ after a protective front cap 14′ has been removed. The medicament delivery device extends along a longitudinal axis AA′ between a proximal end Px′ and a distal end Ds′, wherein the distal end Ds′ is pointing away from the user's body during use of the medicament delivery device 10′. Concerning the terms “distal” or “rear”, and “proximal” of “front”, they refer to points which are further away and closer to the injection site respectively.

The housing 12′ is hollow and may be generally tubular, although other shapes are possible. The housing 12′ extends along the longitudinal axis AA′ between the proximal end Px′ and the distal end Ds′. The housing 12′ comprises a circular (if the housing is tubular) front opening (not visible) closed by the front cap 14′, and a circular (if the housing is tubular) rear opening (not visible) closed by a distal surface 16a′ of a rear cap 16′ (the rear cap 16′ is shown in FIG. 8). The rear cap 16′ is arranged coaxially with the housing 12′, and the distal surface 16a′ of the rear cap 16′ extend orthogonally to the longitudinal axis AA′.

The housing 12′ further comprises an optional window 12a′ through which the user can see the drug to be injected.

The medicament delivery device 10′ comprises the rear cap 16′, depicted in FIG. 8. The rear cap 16′ comprises a distal part 16-D′ and a proximal part 16-P′ removably attached to each other, for instance screwed to each other (as illustrated).

The proximal part 16-P′ of the rear cap 16′ comprises a tubular body 16b′ extending along the longitudinal axis AA′, the tubular body 16b′ being arranged coaxially with the housing 12′ and arranged radially inside a distal portion of the housing 12′. The proximal part 16-P′ of the rear cap 16′ is fixedly attached to the distal portion of the housing 12′, preferably by a pair of flexible tongues 12b′ of the housing 12′ engaging with a pair of corresponding ledges 16c′ on the tubular body 16b′.

The proximal part 16-P′ of the rear cap 16′ further comprises a pair of flexible arms 16d′ attached to the tubular body 16b′ and extending in the proximal direction along the longitudinal axis AA′, able to flex from and to the longitudinal axis AA′ and situated one opposite the other diametrically. The tubular body 16b′ is further equipped with a pair of flexible tongues 16e′ extending in a proximal direction along the axis AA′, able to flex from and to the longitudinal axis AA′ and situated one opposite the other diametrically. The flexible tongues 16e′ of the tubular body 16b′ are displaced on 90° relative to the flexible arms 16d′. Finally, the tubular body 16b′ is equipped, at its proximal end, with a pair of flexible wings 16f′, able to flex from and to the longitudinal axis AA′ and situated one opposite the other diametrically. The flexible wings 16f′ of the tubular body 16b′ are displaced on 90° relative to the flexible arms 16d′.

The function of the flexible arms 16d′, the flexible tongues 16e′ and the flexible 16f′ are not part of the invention, consequently they will not be described here.

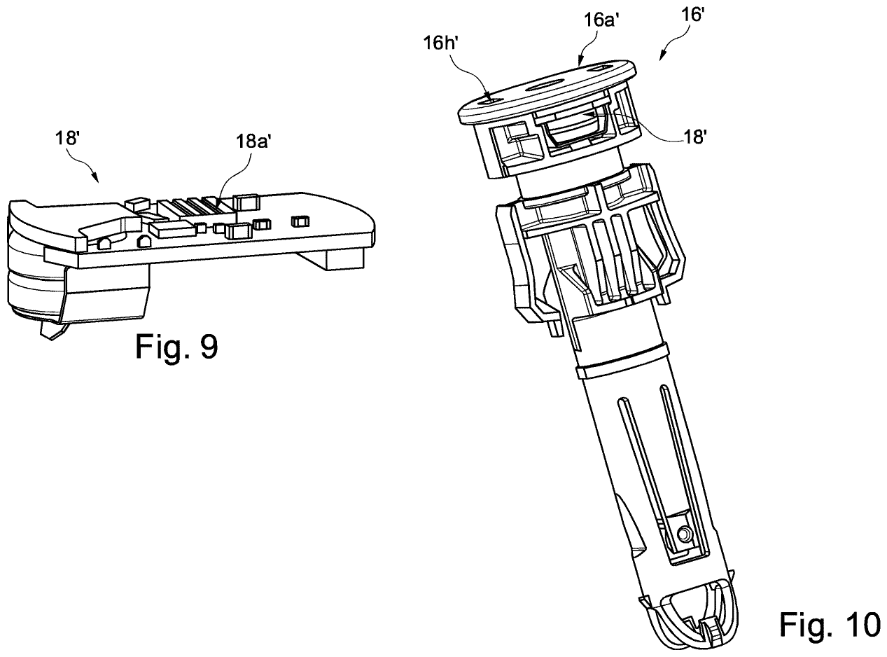

The distal part 16-D′ of the rear cap 16′ comprises the distal surface 16a′ closing the rear opening of the housing 12′. The distal part 16-D′ of the rear cap 16′ further comprises a through slot 17′ extending orthogonal to the longitudinal axis AA′. A tray 18′ (see FIG. 9) can be inserted in the slot 17′ and removed from the slot 17′ through at least one access hole of the slot 17′, orthogonally to the longitudinal axis AA′. In the described example, the slot 17′ has two diametrically opposite access holes (only one is visible).

The slot 17′ is axially located between the distal surface 16a′ and a support plate 16g′ at a proximal extremity of the distal part 16-D′. The distal surface 16a and the support plate 16g′ are supporting elements for the tray 18′.

FIG. 9 shows an exemplary tray 18′ that can be inserted in the slot 17′ of the distal part 16-D′ of the rear cap 16′. The tray 18′ comprises a set of electronic components 18a′, for instance a battery, sensors, indicators and/or data receiving/transmitting means to the medicament delivery devices to provide instructions/indications to the user.

FIG. 10 shows a configuration in which the tray 18′ is arranged in the slot 17′ of the distal part 16-D′ of the rear cap 16′.

As it is visible in FIGS. 8 and 10, the distal surface 16′ of the distal part 16-D′ of the rear cap 16′ has two axial extraction holes in which an extraction tool can be inserted to rotate the distal part 16-D′ relative to the proximal part 16-P′, and thus unscrew the distal part 16-D′ from the proximal part 16-P′.

Consequently, the distal part 16-D′ can be disassembled from the housing 12′, and the tray 18′ can be extracted from the slot 17′ of the distal part 16-D′.

### Third Embodiment

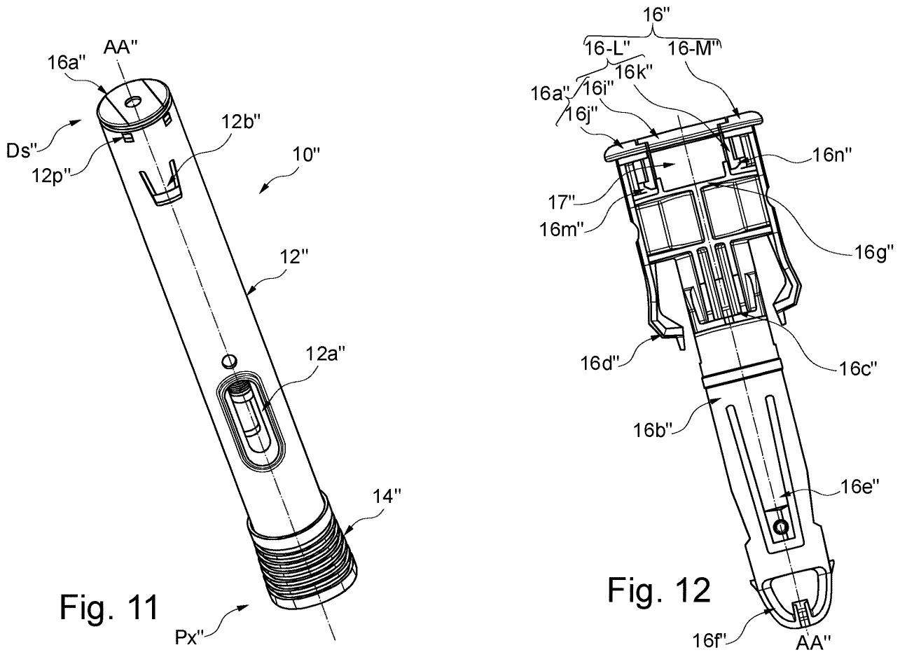

A third embodiment is depicted in FIGS. 11 to 14.

FIG. 11 shows a perspective view of a medicament delivery device 10″. The particular medicament delivery device 10″ illustrated in FIG. 11 is an injector configured to inject a dose of medicament into the body of a user via a syringe arranged in a housing 12″ of the medicament delivery device 10″ after a protective front cap 14″ has been removed. The medicament delivery device extends along a longitudinal axis AA″ between a proximal end Px″ and a distal end Ds″, wherein the distal end Ds″ is pointing away from the user's body during use of the medicament delivery device 10″. Concerning the terms “distal” or “rear”, and “proximal” of “front”, they refer to points which are further away and closer to the injection site respectively.

The housing 12″ is hollow and may be generally tubular, although other shapes are possible. The housing 12″ extends along the longitudinal axis AA″ between the proximal end Px″ and the distal end Ds″. The housing 12″ comprises a circular (if the housing is tubular) front opening (not visible) closed by the front cap 14″, and a circular (if the housing is tubular) rear opening (not visible) closed by a distal surface 16a″ of a rear cap 16″ (the rear cap 16″ is shown in FIG. 12). The rear cap 16″ is arranged coaxially with the housing 12″, and the distal surface 16a″ of the rear cap 16″ extend orthogonally to the longitudinal axis AA″.

The housing 12″ further comprises an optional window 12a″ through which the user can see the drug to be injected.

The medicament delivery device 10″ comprises the rear cap 16″, depicted in FIG. 12. The rear cap 16″ comprises a main body 16-M″ and a lid 16-L″ removably attached to each other, for instance via a snap fit attachment (as illustrated).

The main body 16-M″ of the rear cap 16″ comprises a tubular body 16b″ extending along the longitudinal axis AA″, the tubular body 16b″ being arranged coaxially with the housing 12″ and arranged radially inside a distal portion of the housing 12″. The main body 16-M″ of the rear cap 16″ is fixedly attached to the distal portion of the housing 12″, preferably by a pair of flexible tongues 12b″ of the housing 12″ engaging with a pair of corresponding ledges 16c″ on the tubular body 16b″.

The main body 16-M″ of the rear cap 16″ further comprises a pair of flexible arms 16d″ attached to the tubular body 16b″ and extending in the proximal direction along the longitudinal axis AA″, able to flex from and to the longitudinal axis AA″ and situated one opposite the other diametrically. The tubular body 16b″ is further equipped with a pair of flexible tongues 16e″ extending in a proximal direction along the axis AA″, able to flex from and to the longitudinal axis AA″ and situated one opposite the other diametrically. The flexible tongues 16e″ of the tubular body 16b″ are displaced on 90° relative to the flexible arms 16d″. Finally, the tubular body 16b″ is equipped, at its proximal end, with a pair of flexible wings 16f″, able to flex from and to the longitudinal axis AA″ and situated one opposite the other diametrically. The flexible wings 16f″ of the tubular body 16b″ are displaced on 90° relative to the flexible arms 16d″.

The function of the flexible arms 16d″, the flexible tongues 16e″ and the flexible 16f″ are not part of the invention, consequently they will not be described here.

A most distal surface of the lid 16-L″ of the rear cap 16′, called top surface 16i″ of the lid 16-L″, partly forms the distal surface 16a″ closing the rear opening of the housing 12″. The rest of the distal surface 16a″ is made of most distal surfaces 16″j of the main body 16-M″, on either side of the top surface 16i″ of the lid 16-L″.

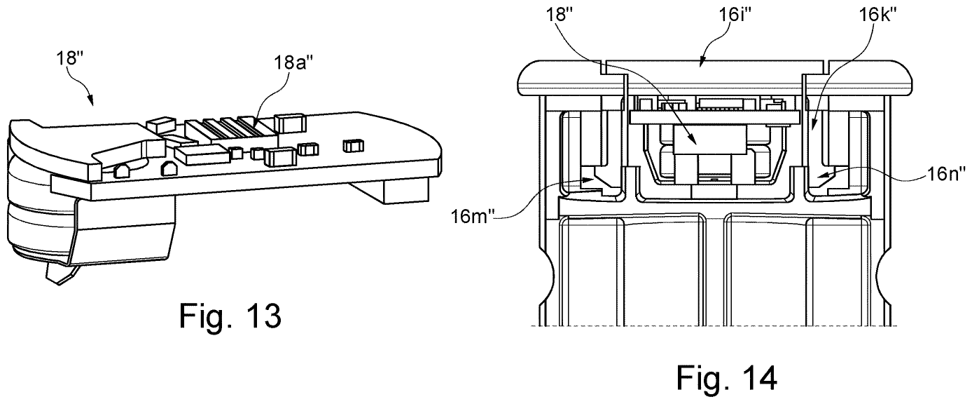

The main part 16-M″ of the rear cap 16″ further comprises a through slot 17″ extending orthogonal to the longitudinal axis AA″. A tray 18″ (see FIG. 13) can be inserted in the slot 17″ and removed from the slot 17″ through at least one access hole of the slot 17″, orthogonally to the longitudinal axis AA″. In the illustrated example, the slot 17″ has two diametrically opposite access holes. The slot 17″ is axially located between the top surface 16i″ of the lid 16-L″ and a support plate 16g″ at a distal extremity of the tubular body 16b″. The top surface 16i″ and the support plate 16g″ are supporting elements for the tray 18″.

In addition to the top surface 16i″, the lid 16-L″ of the rear cap 16″ comprises two flexible arms 16k″. The arms 16k″ extend from the top surface 16i″ in the proximal direction along the longitudinal axis AA′, and are able to flex from and to the longitudinal axis AA′. The arms 16k″ are situated one opposite the other diametrically. The slot 17″ is laterally delimited by the arms 16k″.

Each arm 16k″ of the lid 16-L″ ends with a hook 16n″. The hook 16n″ engages a recess 16m″ in the main body 16.

FIG. 13 shows an exemplary tray 18″ that can be inserted in the slot 17″ of the main part 16-M″ of the rear cap 16″. The tray 18″ comprises a set of electronic components 18a″, for instance a battery, sensors, indicators and/or data receiving/transmitting means to the medicament delivery devices to provide instructions/indications to the user.

FIG. 14 shows a configuration in which the tray 18″ is arranged in the slot 17″ of the main part 16-M″ of the rear cap 16″.

As it is visible in FIG. 11, the distal portion of the housing 12″ comprises two extraction holes 12p″, each facing and connected to a recess 16m″ of the main body 16. Thus, an extraction tool can be inserted in the extraction holes 16p″ to disengage the hooks 16n″ from the recesses 16m″ by flexing the arms 16i″. Consequently, the lid 16-L″ can be disassembled from the main part 16-M″, and the tray 18″ can be extracted from the slot 17′ of the main part 16-M″.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC 500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-15. (canceled)

16. A medicament delivery device, comprising:
a housing extending along a longitudinal axis;
a rear cap closing a rear opening at the distal end of the housing, the rear cap comprising a through slot extending orthogonal to the longitudinal axis, the slot comprising diametrically opposite first and second access holes; and
a tray comprising a set of electronic components, arranged in the slot of the rear cap,
wherein a distal portion of the housing comprises a lateral opening facing the first access hole of the slot of the rear cap, through which the tray can be slid out of the slot.

17. The medicament delivery device of claim 16, wherein the tray comprises a wall closing the lateral opening of the housing.

18. The medicament delivery device of claim 16, wherein the housing comprises at least one cut-out connected to the lateral opening of the housing, said cut-out forming an extraction hole through which an extraction tool can be inserted to slide the tray out of the slot.

19. The medicament delivery device of claim 16, wherein the distal portion of the housing comprises an extraction hole diametrically opposite the lateral opening of the housing and facing the second access hole of the slot of the rear cap, through which an extraction tool can be inserted to push the tray out of the slot.

20. The medicament delivery device of claim 16, wherein the lateral opening and the rear opening are connected to each other at the distal end of the housing.

21. A medicament delivery device, comprising:
a housing extending along a longitudinal axis;
a rear cap closing a rear opening at the distal end of the housing, the rear cap comprising a through slot extending orthogonal to the longitudinal axis, the slot comprising diametrically opposite first and second access holes; and
a tray comprising a set of electronic components, arranged in the slot of the rear cap,
wherein the rear cap comprises a distal part and a proximal part removably attached to each other, the distal part having a distal surface closing the rear opening of the housing, and
wherein the slot is arranged in the distal part of the rear cap.

22. The medicament delivery device of claim 21, wherein the distal surface has at least an extraction hole in which an extraction tool can be inserted to detach the distal part from the proximal part in axial direction.

23. The medicament delivery device of claim 22, wherein the distal part and the proximal part of the reap cap are screwed to each other, and the distal surface of the distal part has two extraction holes in which an extraction tool can be inserted to unscrew the distal part from the proximal part in axial direction.

24. The medicament delivery device of claim 23, wherein the two extraction holes are diametrically opposite relative to the longitudinal axis.

25. The medicament delivery device of claim 21, wherein the proximal part of the rear cap is fixedly attached to a distal portion of the housing.

26. A medicament delivery device, comprising:
a housing extending along a longitudinal axis;
a rear cap closing a rear opening at the distal end of the housing, the rear cap comprising a through slot extending orthogonal to the longitudinal axis, the slot comprising diametrically opposite first and second access holes; and
a tray comprising a set of electronic components, arranged in the slot of the rear cap,
wherein the rear cap comprises a main body and a lid removably attached to each other, the lid having a top surface closing at least partially the rear opening of the housing, and
wherein the slot is distally delimited by the top surface of lid.

27. The medicament delivery device of claim 26, wherein a distal portion of the housing comprises at least an extraction hole through which an extraction tool can be inserted to interact with the lid and detach the lid from the main body.

28. The medicament delivery device of claim 27, wherein the lid comprises two hooks, each hook being engaged in a recess of the main body, and the housing comprises two extraction holes through which an extraction tool can be inserted to disengage the hooks from the recesses.

29. The medicament delivery device of claim 28, wherein the lid comprises two flexible arms, each ending with a hook, the arms extending parallel to the longitudinal axis, the slot being laterally delimited by the arms.

30. The medicament delivery device of claim 26, wherein the slot is proximally delimited by a support plate at a distal extremity of a tubular body of the main body of the rear cap.
